(19) United States
(12) Patent Application Publication
(10) Pub. No.: US 2015/0178873 A1
(43) Pub. Date: Jun. 25, 2015

(54) METHOD AND APPARATUS FOR GENERATING A CLINICAL TRIAL BUDGET

(71) Applicant: Medidata Solutions, Inc., New York, NY (US)

(72) Inventors: Lori Shields, Glenside, PA (US); Joshua Hartman, Conshohocken, PA (US); Theresa Brophy, Oakford, PA (US); Sondra Pepe, Norristown, PA (US)

(73) Assignee: Medidata Solutions, Inc., New York, NY (US)

(21) Appl. No.: 14/136,034

(22) Filed: Dec. 20, 2013

(51) Int. Cl. G06Q 50/22; G06Q 10/06

(52) U.S. Cl. CPC G06Q 50/22; G06Q 10/06313

(57) ABSTRACT

A method for generating a clinical trial budget includes determining a plurality of procedures to be performed during a clinical trial, calculating the likelihood of reimbursement by a third party for each of the procedures, calculating for each of the procedures the expected reimbursement amount, calculating an expected net cost for each of the procedures based on the cost of each procedure and the expected reimbursement amount for each procedure, and summing up the expected net costs of the procedures to determine the clinical trial budget. An apparatus for generating a clinical trial budget is also described and claimed.

### CROSS-REFERENCE TO OTHER APPLICATIONS

This application includes subject matter that overlaps with a pending patent application assigned to the assignee of this application, Medidata Solutions, Inc. That application is entitled, “Method and Apparatus for Determining Complexity of a Clinical Trial,” has attorney docket number 13424616, and is being filed on the same date as this application. The entire disclosure of that patent application is incorporated herein by reference.

### BACKGROUND

Clinical studies or trials to test drugs or devices are very data intensive and can be very expensive. Because patients who are taking part in the studies often suffer from a condition treatable by the drug under test, a number of the procedures, such as x-rays or blood pressure readings, performed on the patients may be reimbursed by a private insurer or Medicare. Reimbursement information is difficult to acquire, so it is often challenging for a sponsor of a clinical trial to determine ahead of time how much a clinical trial may cost.

### BRIEF DESCRIPTION OF THE DRAWINGS

FIG. 1 is a block diagram of a system for generating a clinical trial budget, according to an embodiment of the present invention;

FIGS. 2 and 3 are flowcharts illustrating how budgets for clinical trials may be generated, according to embodiments of the present invention;

FIG. 4 is a table illustrating data to be used to generate a clinical trial budget, according to an embodiment of the present invention; and

FIG. 5 is a diagram illustrating a cost distribution for a clinical procedure, according to an embodiment of the present invention.

Where considered appropriate, reference numerals may be repeated among the drawings to indicate corresponding or analogous elements. Moreover, some of the blocks depicted in the drawings may be combined into a single function.

### DETAILED DESCRIPTION

In the following detailed description, numerous specific details are set forth in order to provide a thorough understanding of embodiments of the invention. However, it will be understood by those of ordinary skill in the art that the embodiments of the present invention may be practiced without these specific details. In other instances, well-known methods, procedures, components, and circuits have not been described in detail so as not to obscure the present invention.

A sponsor, such as a drug manufacturer, of a clinical drug trial or a contract research organization (CRO) that undertakes a clinical trial for a sponsor often would like to know beforehand how much a clinical trial will cost. (Hereinafter, the word “sponsor” means sponsor or CRO or any other entity or person who plans or runs a clinical trial.) But that determination is typically a combination of how much the procedures performed in the study will cost as well as what reimbursements are made by third parties (hereinafter, the term “third party” means a private or governmental insurer (such as Medicare or Medicaid) and includes any entity that pays for a procedure other than the patient, the doctor, or the sponsor). Before a study is undertaken, a sponsor will typically negotiate with study sites to determine how much the sites will get paid for performing certain procedures, and knowledge of the likelihood that a certain procedure will be reimbursed by a third party allows the sponsor to negotiate more ably as well as better predict what the trial itself will cost. Underlying the negotiations are laws, e.g., in 42 CFR Parts 402 and 403, that prevent sites from being paid twice for the same procedure—once by a sponsor and once by a third party.

Currently, sponsors may employ people who attempt to make such budget determinations, but are limited because they have access to only the data relevant to their own employer and do not have a comprehensive view of reimbursement data across the industry. Sponsors may also work with sites to attempt to make such determinations, but these sites may not have the global view needed to determine reimbursement data for the whole study.

The present invention may be used to determine a budget for a clinical trial by taking into account third-party reimbursement. This third-party reimbursement is often called “routine cost coverage” or “standard of care,” and typically encompasses the care that would have been provided to the patient and reimbursed in the absence of the clinical trial. It is often difficult, however, to accurately gauge routine cost coverage because reimbursement depends not only on the procedure, but also the patient, the patient's insurer, and the particular disease. For example, a chest x-ray may be reimbursed differently (different frequency, different intervals, and different amounts) depending on whether the patient is covered by private insurers, such as a PPO (preferred provider organization) or an HMO (health maintenance organization), or a government-sponsored plan, such as Medicaid, Medicare, and Tricare (formerly CHAMPUS) and CHAMPVA (for military and veterans and their families). In addition, a course of treatment may have an effect on the reimbursement availability and amount; for example, a well-studied disease like hypertension may have stable reimbursement policies whereas reimbursement varies for a disease like diabetes that has more variability than hypertension.

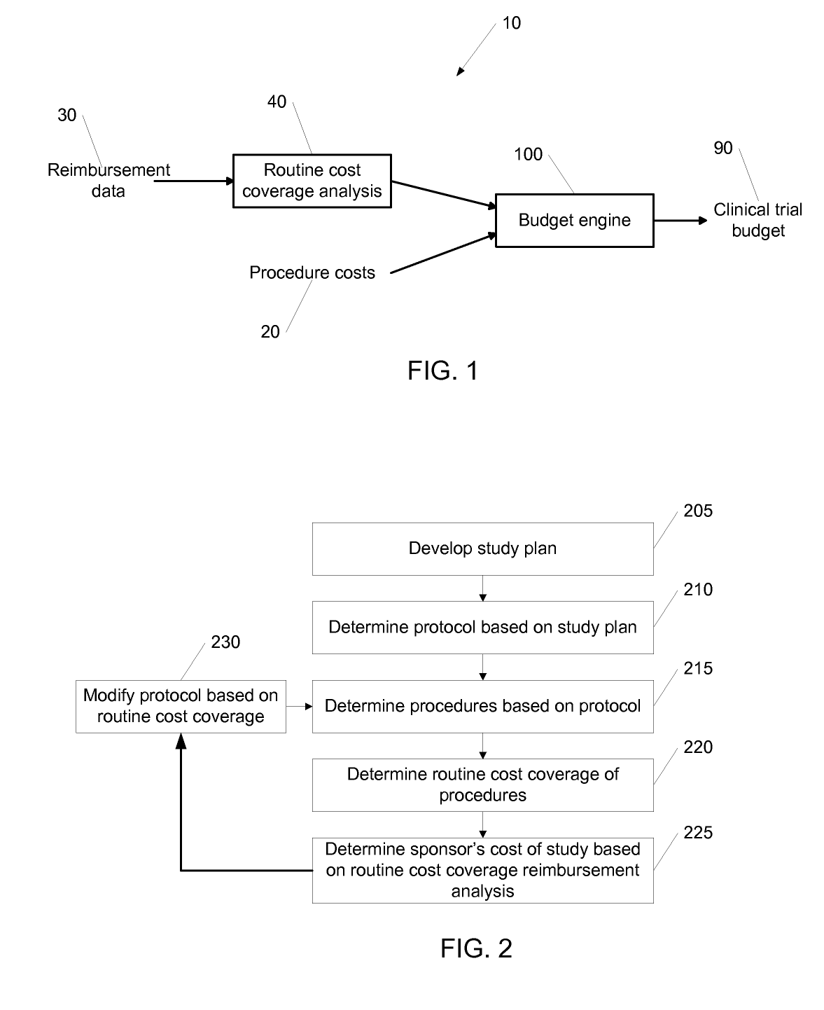

Reference is now made to FIG. 1, which is a block diagram of a budget system 10 according to an embodiment of the present invention. Broadly speaking, in FIG. 1, budget engine 100 takes as inputs procedure costs 20 and routine cost coverage analysis 40, which has as an input reimbursement data 30, and outputs clinical trial budget 90. Such reimbursement data may come from, for example, public databases, sponsors of clinical trials, health insurers, or private data suppliers.

The blocks shown in FIG. 1 are examples of modules that may comprise budget system 10, and do not limit the blocks or modules that may be part of or connected to or associated with budget system 10. For example, budget engine 100 may perform routine cost coverage analysis 40, or routine cost coverage analysis block 40 may perform analyses other than just routine cost coverage. The blocks in FIG. 1 may generally be implemented in software or hardware or a combination of the two.

Budget system 10 may be implemented on a standalone computer or on a network, for example, over the Internet as a cloud-based service or hosted service, which may be accessed through a standard web service application programming interface (API).

FIG. 2 is a flowchart illustrating an embodiment of the present invention, showing one way for a sponsor to develop a budget for a clinical trial. In operation 205, the sponsor may develop a “study plan” for the clinical trial or study. Such a plan may be an outline of the study design which may be customized to fit the final protocol design. In operation 210, the sponsor may then determine a protocol based on the study plan. Such a protocol may include a synopsis, objectives, schedule of activities, statistical plan and outcome measures. In operation 215, the sponsor may determine which medical or clinical procedures may be performed based on the study protocol. Such procedures may include x-rays, CT scans, blood draws, readings of vital signs, office consultations—likely any procedure for which there is a medical procedure code, one class of which are “CPT Codes” (“CPT” stands for “current procedural terminology”), which are maintained by the American Medical Association.

In operation 220, routine cost coverage of the procedures identified in operation 215 may be performed. This analysis may involve calculating summary statistics to determine how frequently a procedure is performed for a diagnosis group. The analysis may then calculate summary statistics to determine the typical interval between performances of a procedure for a diagnosis group. The analysis may also take into account the percentages of the population (or percentage of the diagnosis group, if available) that have certain types of insurance, e.g., private PPO/HMO, Medicaid, Medicare, Tricare, or CHAMPVA, knowing that different insurers may offer reimbursement at different rates and for different amounts, and that rates and amounts may vary based on the disease to be treated.

Operation 225 then determines the sponsor's cost of study based on the routine cost coverage reimbursement analysis. At its foundation, this may involve subtracting the expected reimbursement from the cost of the procedures. In this scenario, a budget (e.g., net cost) may be found by:

(# of procedures)*(average cost of procedures)−(# of procedures reimbursed)*(average reimbursement amount) (Eq.1)

If there are 30 procedures with an average cost of $200 and 20 of the procedures are reimbursed at an average reimbursement of $150, the net cost will be $6000−$3000=$3000.

Because the routine cost coverage may not be exactly known, operation 225 may involve statistical analysis to determine the expected or probable cost of the study. Examples of one type of statistical analysis are shown in Table 1 below, in which the cost of the procedures remains $6000, but the percentage of procedures reimbursed and the probability of reimbursement may vary. Row 1 shows the previous example in terms of this probability, in which the probability of 67% of the procedures (or 20 out of 30) being reimbursed is 100%. Row 2 shows how expected net cost may change if the probability of 67% of the procedures being reimbursed is 80%. If the average reimbursement is $150 as before, the total reimbursement is found by multiplying the two percentages and the average reimbursement for a total of 30*67%*80%*150=$2412, and net cost rises to $3588.

TABLE 1

| | Average | | % of | Probability | Average | | |
|---|---|---|---|---|---|---|---|
| # | cost/ | | procedures | of | reimbursement | Total | Expected |
| procedures | procedure | Total cost | reimbursed | reimbursement | amount | reimbursement | net cost |
| 30 | 200 | 6000 | 67% | 100% | 150 | 3000 | 3000 |
| 30 | 200 | 6000 | 67% | 80% | 150 | 2412 | 3588 |
| 30 | 200 | 6000 | 50% | 90% | 150 | 2025 | 3975 |
| 30 | 200 | 6000 | 25% | 100% | 150 | 1125 | 4875 |

As the percentage of procedures reimbursed decreases, the probability of reimbursement likely increases, and the expected net cost will change. In the case of Table 1, if the probability of 50% of the procedures (or 15 out of 30) being reimbursed is 90%, the expected net cost would rise to $3975.

There may typically be a point where the probability of reimbursement approaches 100%. If that occurs for 25% of the procedures (or 7.5 out of 30), then the expected net cost of the study would rise to $4875, as shown in the last row of Table 1.

Once the cost of the study in operation 225 is determined, a feedback loop may be introduced into flowchart of FIG. 2, as shown in operation 230. In this operation, the sponsor may modify the protocol based on the routine cost coverage analysis in order to maximize the expected reimbursement and thus minimize the expected budget for the study. For example, the study design may originally call for a procedure to be performed four times, but the routine cost coverage analysis may show that the procedure will only be reimbursed twice for a patient during the study. In that case, the designer may modify the protocol to call for the procedure to be performed only two times, in order to maximize the likelihood of reimbursement.

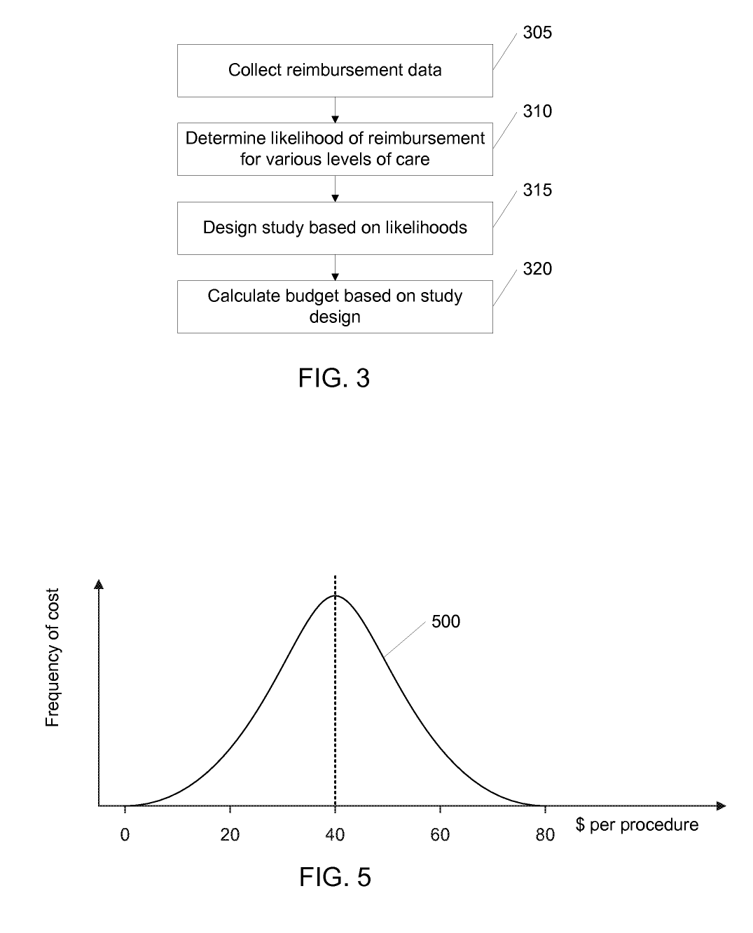

A flowchart that takes into account the probability (or likelihood) of reimbursement in Table 1 is shown in FIG. 3. Reimbursement data may be collected in operation 305. In operation 310, the likelihood of reimbursement may be determined for various levels of care. Then, in operation 315, a study may be designed based on the likelihoods of reimbursement for different procedures. Finally, a budget may be calculated based on the study design. In this way, a study may be designed to take advantage of routine cost coverage, which may provide the highest potential cost savings to a sponsor.

Besides the operations shown in FIGS. 2 and 3, other operations or series of operations are contemplated to determine a clinical trial budget. Moreover, the actual orders of the operations in the flow diagrams are not intended to be limiting, and the operations may be performed in any practical order. For example, operation 230 may involve modifying the study plan, which, in turn may modify the protocol, in which case the output of operation 230 may be input to operation 210 rather than 215. In other embodiments, there may be other feedback processes within individual operations, such that the study plan or protocol is designed with some knowledge of the routine cost coverage, along the lines of the flowchart in FIG. 3, which may reduce the need for an explicit feedback loop in operation 230.

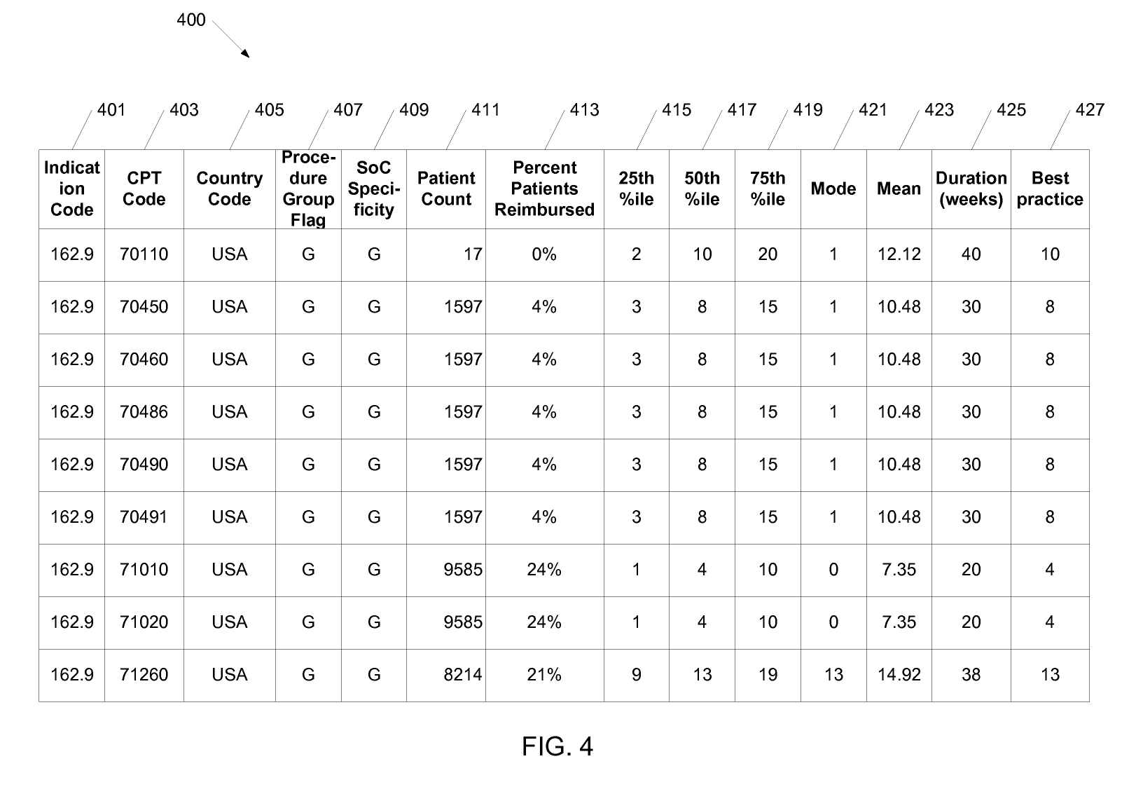

Another way of examining the expected net cost (or, alternatively, the expected reimbursement) is provided in FIG. 4, which shows table 400 that collects numerous data according to an embodiment of the present invention. The patients whose data appear in table 400 have the indication (disease) noted in column 401—indication code (also called “ICD-9” code) 162.9 is malignant neoplasm of bronchus and lung, which may be associated with lung cancer. In another embodiment, table 400 may include a column with ICD-10 codes that indicate what occurrence of the disease in column 401 the patient has (e.g., 1st, 2nd, 3rd, etc.). As shown in FIG. 4, table 400 includes nine rows, each of which indicates a different procedure, as shown by the CPT code in column 403, that may be associated with this disease. The nine CPT codes in table 400 are radiological procedures such as chest X-rays and face, neck, head, and chest CT (computed tomography) scans.

Column 405 indicates the country in which the procedure takes place; the invention includes the ability to generate budgets on a country-by-country basis as well as on a finer geographical scale, such as by region or state or ZIP code. Column 407, Procedure Group Flag, indicates the degree of match for a specific procedure (similar to a 3-digit ZIP code roll-up). Related procedures may be grouped when medically relevant. Column 409, SoC Specificity, represents the level of match for each diagnosis, procedure, and country combination (e.g., G=Indication Group, I=Specific Indication). Column 411, Patient Count, indicates how many patients are in a diagnosis and geographic group over a defined interval of time. Column 413 indicates the percentage of patients for whom the procedure in column 403 was reimbursable, which indicates the likelihood of routine cost coverage, which may vary significantly across diagnosis groups. The number in column 413 may be related to the probability of reimbursement in Table 1, and the flowchart in FIG. 3 illustrates a way to use the information in column 413 to design a clinical study and calculate a study budget. Columns 415, 417, 419 indicate the 25th, 50th, and 75th percentiles, respectively, as to the frequency with which that procedure was reimbursed within the time period shown in column 425. Column 421, Mode, indicates the most prevalent number of times the procedure was reimbursed within the time period. Column 423, Mean, indicates the average number of times the procedure was reimbursed within the time period.

As an example of how the data in table 400 may be used, the last row of the shows that the procedure has CPT code 71260, which is a chest CT scan. There were 8214 patients in this diagnosis, geography, and time interval who had this procedure performed, of which 21% had the procedure reimbursed within 38 weeks (column 425). Column 421 (Mode) shows that the most prevalent number of times the procedure was reimbursed was 13 times per patient over the 38 weeks (approximately once every three weeks). Column 423 shows that the average patient having the procedure reimbursed had almost 15 (14.92) chest CT scans reimbursed over that time period. Columns 415, 417, and 419 in the last row indicate that 25% of the patients that were reimbursed were reimbursed 9 or fewer times, 50% of the patients that were reimbursed were reimbursed 13 or fewer times, and 75% of the patients that were reimbursed were reimbursed 19 or fewer times. In table 400, column 427 (best practice) is the same as column 421 (mode), but it can be any value that is determined by system 10 to be the best indication of how many times a procedure would be reimbursed. For example, the system may define a default threshold for which it considers a procedure to be routine. Users (e.g., sponsors) may adjust the threshold based on the data and their experience as necessary. This information may then be fed back to the system to fine tune the best practice value, and may be subject to updating based on operational data. It is this best practice value that may be used to estimate the routine cost coverage to determine a budget for a clinical trial.

The present invention may used to help sponsors negotiate with study sites over payments, as shown in the following example. Embodiments of the present invention also include using the data to determine likely costs for a procedure, for example a blood draw. This may be represented as shown in FIG. 5 by a cost distribution 500 that has $20 at the 25th percentile, $40 at the 50th percentile, and $60 at the 75th percentile (which means that 25% of the procedures cost $20 or less, 50% of the procedures cost $40 or less, and 75% of the procedures cost $60 or less). A sponsor may budget a cost of $40 for each blood draw, and there may be 10 blood draws per patient in a study. Thus, the total cost to the sponsor would be $400. However, if the routine cost coverage analysis shows that two procedures may be reimbursed, the net cost to the sponsor would be $320 per patient.

In a negotiation with a site over how much the site would get paid to perform a blood draw, the sponsor can use this data as an indication of what would be reasonable for a site to be paid. However, the site may try to negotiate a higher price (even if the site did not have the same reimbursement or cost information). If the site wants to be paid $45 per procedure, the sponsor, taking into account routine cost coverage of two procedures, would end up paying $360. Note that this is $40 less than the possible cost for the procedures at the lower cost, but without accounting for routine cost coverage. In this case, the sponsor was in a better position to negotiate and had a better idea of what it would have to pay, but, more importantly, budget, for the procedures, thus freeing up $40 per patient to be spent by the sponsor on other procedures or studies.

The previous embodiments are described in the setting of generating budgets for clinical trials, including clinical trials for drugs or medical devices. It is understood, however, that embodiments of the invention can be used in other fields involving clinical study feasibility, patient selection, site identification, and patient tolerance for a study design.

One benefit of the present invention is that a sponsor can more accurately understand the current standard of care in a patient population so that it can design a clinical study for the best analysis of their new product against the best current treatment, making it more likely a regulatory agency such as the U.S. Food and Drug Administration (FDA) will approve the drug. Another benefit is that the present invention allows the sponsor to better estimate its clinical trial budget and clinical trial cash flow so that it may support more concurrent studies or more sites for its studies. A further benefit is that a sponsor can be better equipped to negotiate payment arrangements with the study sites.

The present invention differs from other systems that may provide budgeting assistance for clinical trials. For example, those systems may not have access to comprehensive reimbursement data or procedure costs. Those systems may lack the knowledge regarding the likelihood or frequency with which medical procedures are reimbursed. Moreover, those systems may not be able to identify potential for reimbursement in a given geography and may not be designed to learn from operational data over time.

Aspects of the present invention may be embodied in the form of a system, a computer program product, or a method. Similarly, aspects of the present invention may be embodied as hardware, software or a combination of both. Aspects of the present invention may be embodied as a computer program product saved on one or more computer-readable media in the form of computer-readable program code embodied thereon.

For example, the computer-readable medium may be a computer-readable signal medium or a computer-readable storage medium. A computer-readable storage medium may be, for example, an electronic, optical, magnetic, electromagnetic, infrared, or semiconductor system, apparatus, or device, or any combination thereof.

A computer-readable signal medium may include a propagated data signal with computer-readable program code embodied therein, for example, in baseband or as part of a carrier wave. Such a propagated signal may take any of a variety of forms, including, but not limited to, electromagnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device.

Computer program code in embodiments of the present invention may be written in any suitable programming language. The program code may execute on a single computer, or on a plurality of computers. The computer may include a processing unit in communication with a computer-usable medium, wherein the computer-usable medium contains a set of instructions, and wherein the processing unit is designed to carry out the set of instructions.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

1. A method for generating a clinical trial budget, comprising:
determining a plurality of procedures to be performed during a clinical trial, each of the procedures having a cost;
calculating a likelihood of reimbursement by a third party for each of the procedures;
calculating for each of the procedures an expected reimbursement amount based on the likelihood of reimbursement;
calculating an expected net cost for each of the procedures based on the cost of said each procedure and the expected reimbursement amount for said each procedure; and
summing up the expected net costs of the procedures to determine the clinical trial budget.

2. The method of claim 1, further comprising modifying the procedures based on the likelihood of reimbursement for each of the procedures.

3. The method of claim 2, wherein the modification of the procedures is based on minimizing the clinical trial budget.

4. The method of claim 1, further comprising calculating the frequency that each procedure is expected to be reimbursed during the clinical trial.

5. The method of claim 1, further comprising calculating the interval between performances of each procedure during the clinical trial.

6. The method of claim 1, further comprising collecting reimbursement data related to past performance of each of the procedures to calculate the likelihood of reimbursement and the expected reimbursement amount for each procedure.

7. The method of claim 6, wherein the reimbursement data comes from one or more public databases.

8. The method of claim 6, wherein the reimbursement data comes from one or more sponsors of clinical trials.

9. The method of claim 1, wherein calculating a likelihood of reimbursement by a third party for each of the procedures comprises using data from the country in which the clinical trial will be held.

10. A method for calculating an expected net cost of a clinical trial procedure, the procedure having a cost, the method comprising:
calculating a likelihood of reimbursement by a third party for the procedure;
calculating an expected reimbursement amount based on the likelihood of reimbursement; and
calculating the expected net cost of the procedure based on the cost of the procedure and the expected reimbursement amount.

11. The method of claim 10, further comprising collecting reimbursement data related to past performance of the procedure to calculate the likelihood of reimbursement and the expected reimbursement amount.

12. The method of claim 11, wherein the reimbursement data comes from one or more public databases.

13. The method of claim 11, wherein the reimbursement data comes from one or more sponsors of clinical trials.

14. An apparatus for generating a clinical trial budget, comprising:
a routine cost coverage analysis engine configured to:
collect reimbursement data related to past performance of a plurality of procedures to be performed during a clinical trial, each of the procedures having a cost;
calculate a likelihood of reimbursement by a third party for each of the procedures; and
calculate for each of the procedures an expected reimbursement amount based on the likelihood of reimbursement; and
a budget engine configured to:
calculate an expected net cost for each of the procedures based on the cost of said each procedure and the expected reimbursement amount for said each procedure; and
sum up the expected net costs of the procedures to determine the clinical trial budget.

15. The apparatus of claim 14, wherein the reimbursement data comes from one or more public databases.

16. The apparatus of claim 14, wherein the reimbursement data comes from one or more sponsors of clinical trials.

17. The apparatus of claim 14, wherein the budget engine determines the procedures to be performed based on the likelihood of reimbursement for each of the procedures.

18. The apparatus of claim 14, wherein the routine cost coverage analysis engine calculates the likelihood of reimbursement for each of the procedures based on the statistical mode of the frequency of reimbursement of said each procedure.

19. The apparatus of claim 14, wherein the routine cost coverage analysis engine calculates the likelihood of reimbursement for each of the procedures based on the statistical mean of the frequency of reimbursement of said each procedure.
